(19) United States
(12) Patent Application Publication
(10) Pub. No.: US 2025/0152835 A1
(43) Pub. Date: May 15, 2025

(54) DOSE DELIVERY DEVICE WITH MODIFIED DECOUPLING CLUTCH

(71) Applicant: Phillips-Medisize A/S, Struer (DK)

(72) Inventor: Peter Lundholm Jensen, Copenhagen (DK)

(21) Appl. No.: 18/836,330

(22) PCT Filed: Jan. 12, 2023
(86) PCT No.: PCT/IB2023/050295

§ 371 Date: Aug. 6, 2024

(30) Foreign Application Priority Data
Jan. 14, 2022 (EP) 22151559.6

(51) Int. Cl. A61M 5/315

(52) U.S. Cl. CPC A61M 5/31551

(57) ABSTRACT

A rotatable dose selector clutch for a dose delivery device is disclosed. The dose delivery device having a dosage scale drum with interior dosage scale coupling means and a thrust bearing with exterior thrust bearing coupling means, the thrust bearing is configured to be threaded coupled to a piston rod, wherein the dose selector clutch is configured to be arranged in: •a coupled position, wherein the dose selector clutch couples to the dosage scale drum and the thrust bearing, such that the dosage scale drum and the thrust bearing rotates when the dose selector clutch is rotated, and •a decoupled position, wherein the dose selector clutch decouples from the dosage scale drum and the thrust bearing, such that the dosage scale drum and the thrust bearing are free to rotate independently of the dose selector clutch.

### RELATED APPLICATIONS

This application claims priority to European Patent Application No. 22151559.6, filed Jan. 14, 2022, which is incorporated herein by reference in its entirety.

### FIELD OF THE INVENTION

The invention relates to a rotatable dose selector clutch/a rotatable dose selector/a push button configured to be arranged in a coupled position for coupling and decoupling a dosage scale drum and a thrust bearing in a dose delivery device, and a decoupled position for decoupling the dosage scale drum and the thrust bearing in the dose delivery device. The invention further relates to the dose delivery device.

### BACKGROUND

A number of dose delivery devices are known in which a dose of medicament to be delivered to a patient is set by rotation of a tubular part and the subsequent delivery of the dose of medicament is obtained by pressing an injection button in a delivery direction. From EP 2 618 872 is known a dose delivery device wherein a dose can be set by rotating a dose setting member, whereby a push button is elevated from one end of the device, and the set dose can then be injected by pressing the push button back to its non-elevated position, thereby moving a lead screw co-operating with the piston in a cartridge and expelling a medicament out of the cartridge through a needle. FIG. 2 of EP 2 618 872 discloses an embodiment, where there is a gearing mechanism with a helical track between a first gearing member and a driver. After a dose has been set, a dose dial rotationally coupled to the dose setting member in a dose setting configuration, is exposed. This has been found to introduce a risk when a user by mistake grabs and starts to rotate the dose dial backwards while applying just enough pressure to the push-button for the dose setting member and the dose dial to decouple. This causes an incorrect dose to be displayed on the dose dial, whereby a user may receive an incorrect dose of medicament. Further, during the dose delivery process when the push button is pressed in the proximal direction, the helical track coupling causes the dose setting member to rotate slowly, which may further induce a user discomfort.

It is an objective of the invention to provide a dose delivery device with an improved security.

### SUMMARY

Disclosed herein in a first aspect is a rotatable dose selector clutch for a dose delivery device. The dose delivery device is having a dosage scale drum with interior dosage scale coupling means and a thrust bearing with exterior thrust bearing coupling means, the thrust bearing is configured to be threaded coupled to a piston rod.

The dose selector clutch is configured to be arranged in:
a coupled position, wherein the dose selector clutch couples to the dosage scale drum and the thrust bearing, such that the dosage scale drum and the thrust bearing rotates when the dose selector clutch is rotated, and
a decoupled position, wherein the dose selector clutch decouples from the dosage scale drum and the thrust bearing, such that the dosage scale drum and the thrust bearing are free to rotate independently of the dose selector clutch.

The dose selector clutch comprises inner coupling means and outer coupling means arranged along an annular circumference, the annular circumference extending in a rotational plane perpendicular to a centre axis extending in an axial direction, the inner and outer coupling means protrude from the annular circumference with the inner coupling means protruding towards the centre axis and the outer coupling means protruding away from the centre axis.

By the thrust bearing being configured to be threaded coupled to a piston rod is meant that the thrust bearing is either indirectly threadedly coupled to the piston rod or directly threadedly coupled to the piston rod.

Disclosed herein in a second aspect is a rotatable dose selector for a dose delivery device, the dose delivery device having a dosage scale drum with interior dosage scale coupling means and a thrust bearing with exterior thrust bearing coupling means, the thrust bearing is configured to be threaded coupled to a piston rod.

The dose selector is configured to be arranged in:
a coupled position, wherein the dose selector couples to the dosage scale drum and the thrust bearing when the dose selector is rotated, such that the dosage scale drum and the thrust bearing rotates when the dose selector is rotated,
a decoupled position, wherein the dose selector decouples from the dosage scale drum and the thrust bearing, such that the dosage scale drum and the thrust bearing are free to rotate independently of the dose selector.

The dose selector comprises a clutch comprising inner coupling means and outer coupling means arranged along an annular circumference, the annular circumference extending in a rotational plane perpendicular to a centre axis extending in an axial direction, the inner and outer coupling means protrude from the annular circumference with the inner coupling means protruding towards the centre axis and the outer coupling means protruding away from the centre axis.

In the coupled position, the outer coupling means are configured to interact with the dosage dial coupling means, and the inner coupling means are configured to interact with the thrust bearing coupling means, when the dose selector is rotated.

By the thrust bearing being configured to be threaded coupled to a piston rod is meant that the thrust bearing is either indirectly threadedly coupled to the piston rod or directly threadedly coupled to the piston rod.

Disclosed herein in a third aspect is a push button for a dose delivery device having a dosage scale drum with interior dosage scale coupling means and a thrust bearing with exterior thrust bearing coupling means, the thrust bearing is configured to be threaded coupled to a piston rod.

The push button is configured to be arranged in:
a coupled position, wherein the push button couples to the dosage scale drum and the thrust bearing when the push button is rotated, such that the dosage scale drum and the thrust bearing rotates when the push button rotates,
a decoupled position, wherein the push button decouples from the dosage scale drum and the thrust bearing, such that the dosage scale drum and the thrust bearing are free to rotate independently of the push button.

The push button comprises a clutch comprising inner coupling means and outer coupling means arranged along an annular circumference, the annular circumference extending in a rotational plane perpendicular to a centre axis extending in an axial direction, the inner and outer coupling means protrude from the annular circumference with the inner coupling means protruding towards the centre axis and the outer coupling means protruding away from the centre axis,

In the coupled position, the outer coupling means are configured to interact with the dosage scale coupling means, and the inner coupling means are configured to interact with the thrust bearing coupling means, when the push button rotates.

By the thrust bearing being configured to be threaded coupled to a piston rod is meant that the thrust bearing is either indirectly threadedly coupled to the piston rod or directly threadedly coupled to the piston rod.

By either of the rotatable dose selector clutch according to the first aspect, the rotatable dose selector according to the second aspect, or the push button according to the third aspect is obtained an improved double coupling mechanism, which allows for simultaneous coupling to and decoupling from both a dosage scale drum and a thrust bearing by movement of only the rotatable dose selector clutch according to the first aspect, or the rotatable dose selector according to the second aspect, or the push button according to the third aspect. The simultaneous decoupling of the rotatable dose selector clutch according to the first aspect, or the rotatable dose selector according to the second aspect, or the push button according to the third aspect ensures that during dose delivery, it may be prevented that the user by mistake reset the dose resulting incorrect dose delivery. Further, as the thrust bearing and the dosage scale can be simultaneously decoupled, it can be obtained that all driver parts of the dose delivery device can rotate independently of the dose selector clutch and the dosage scale during dose delivery. Thus, there is a free rotation of the dosage selector during dosing. This improves the user experience and comfort when using the device. Further, the configuration with one part couples/decouples to two other parts, allows for more robust dose delivery device comprising fewer parts.

This is an improvement over known drug delivery devices such as disclosed in either of the documents US 2016/287803 A1 and US 2015/224266 A1.

In US 2016/287803 A1, the number sleeve (item 24 functioning as a dosage scale drum) and the clutch (item 26) are couple during manufacturing in such a manner, which does not allow the two parts to decouple later during e.g. injection as outlined in [0081]. Thus, there is no de-coupled position, where the number sleeve and the clutch decouples as the two parts are inseparably locked during manufacturing.

In US 2015/224266 A1, the clicker part (item 102 functioning as a thrust bearing) and the clutch (item 90) decouples from the dial sleeve (item 62 part of item 60 functioning as a dosage scale drum) during dosing. However, the clicker part comprises a set of the teeth, which prevents a simultaneously decoupling of the clutch and the clicker part. In fact, the clutch and the clicker part move rotationally together during dosing, i.e. there is no double decoupling as specified in the rotatable dose selector clutch according to the first aspect, the rotatable dose selector according to the second aspect, or the push button according to the third aspect.

In one or more examples, the coupled position is adapted for dose setting operation of the dose delivery device and the decoupled position is adapted for dose delivery operation of the dose delivery device. Alternatively, the decoupled position may be adapted for dose setting operation of the dose delivery device and the coupled position may be adapted for dose delivery operation of the dose delivery device.

In one or more examples, the inner coupling means, the outer coupling means or both comprises spines and/or ratchet teeth.

The inner coupling means and the outer coupling means may protrude in the same plane in opposite directions. Alternatively, the inner coupling means and the outer coupling means may be positioned in parallel planes preferably separated by a smaller distance in the longitudinal direction.

The inner coupling means and the outer coupling means may protrude inwardly and outwardly, respectively, in a configuration where the inner coupling means and the outer coupling means are positioned opposite each other. Alternatively, the inner coupling means may protrude inwardly and the outer coupling means may protrude outwardly in an alternating configuration, where each inwardly protruding coupling means is positioned between two outer coupling means protruding outwardly.

In one or more examples, the dose selector clutch comprises a second set of outer coupling means configured for interacting with a rotational dose selector for setting the dose.

In one or more examples, the dose selector clutch is part of a rotational dose selector for setting the dose.

In one or more examples, the dose selector clutch comprises an annular body extending along the annular circumference with the inner and outer coupling means, protruding from the annular body.

In one or more examples, the rotatable dose selector comprises an annular body extending along the annular circumference with the inner and outer coupling means, protruding from the annular body.

In one or more examples, the annular body is formed as a tubular part with an aperture, the tubular part is adapted to receive at least a part of the thrust bearing through the aperture in the axial direction, and the inner and outer coupling means are arranged to protrude from the tubular part. The tubular part may alternatively or additionally comprise a number of cut-outs. Thereby, the tubular part becomes partly interrupted/opened. The cut-out may be present on only in a selected part of the tubular part, such as a lower part of the tubular part and/or an upper part of the tubular part.

In one or more examples, the inner coupling means, the outer coupling means or both are arranged on a first half part of the tubular part being closest to the aperture adapted to receive at least a part of the thrust bearing.

In one or more examples, the tubular part comprises an inner surface and an outer surface, wherein the inner coupling means protrude from the inner surface, and the outer coupling means protrude from the outer surface.

A multiple of inner coupling means and/or a multiple of outer coupling means may be positioned around the annular body in the radial direction with a distribution providing approximately the same distance between any two consecutive inner coupling means and/or outer coupling means. Alternatively, the multiple of inner coupling means and/or a multiple of outer coupling means may be grouped in smaller subgroups of coupling means. In such configuration, the distance between any two pair of consecutive inner coupling means and/or any two pair of consecutive outer coupling means will vary between the different consecutive pairs of inner coupling means and/or outer coupling means.

In one or more examples, the rotatable dose selector comprises an outer surface for being manually rotatable operated for setting a dose of the dose delivery device. A user may set a dose in a dose delivery device by rotation of the rotatable dose selector by grapping the outer surface of the dose delivery device and rotating the dose selector.

Disclosed herein in a fourth aspect is a dose delivery device with a rotatable dose selector clutch according to the above, wherein the dose delivery device further comprises:
a housing;
a tower;
a dosage scale drum comprising dosage scale coupling means;
a thrust bearing comprising thrust bearing coupling means;
a piston rod arranged in a longitudinal axial direction and rotationally locked to the housing and in threaded engagement with the thrust bearing allowing axial movement of the thrust bearing relative to the housing.

By the piston rod being in threaded engagement with the thrust bearing is meant that the thrust bearing is either in an indirect threadedly engagement with the piston rod or a direct threaded engagement with the piston rod. An indirect threaded engagement may be an engagement by means of a direct engagement of other parts.

The dose delivery device may be configured to be operated in:
a dose setting mode with the rotatable clutch arranged in a coupled position, wherein said outer coupling means are configured to interact with the dosage dial coupling means, and said inner coupling means are configured to interact with the thrust bearing coupling means, when the dose selector clutch is rotated, such that the dosage scale drum, the thrust bearing and the dose selector clutch are rotationally locked to each other and are axially displaced relative to the piston rod in a distal direction, such that a dosage is set by the thrust bearing's axial position on the piston rod, and
a dose delivery mode with the rotatable dose selector clutch arranged in a decoupled position, wherein the dose selector clutch rotationally decouples from the dosage scale drum and the thrust bearing, and wherein the thrust bearing is axially displaceable in a proximal direction, such that the set dose is delivered by axially displacing the piston rod.

The dose delivery device may further comprise:
a rotatable dose selector, and
an axially moveable push button in axial connection with the thrust bearing.

In one or more examples, the dose selector is configured to couple to the clutch at least during the dose setting mode. The dose delivery device may be configured for being in the dose setting mode when rotating the dose selector in a rotational setting direction.

In one or more examples, the dose selector is configured to couple to the clutch during a dose correction mode. The dose delivery device may be configured for correcting a dose in the dose correction mode. The dose delivery device may be configured for being in the dose correcting mode when rotating the dose selector in a rotational correcting direction. The rotational correcting direction may be the opposite rotational direction of the rotational setting direction.

The push button may be configured to interact with the clutch during the dose delivery mode to displace the clutch in an axial direction from the coupled position to the decoupled position. Alternatively, the push button may be part of the clutch. If the push button is part of the clutch, it may be so with a flexible connection between the push button and the clutch.

In one or more examples, the push button is configured to interact with the clutch during the dose delivery mode to displace the clutch in an axial direction from the coupled position to the decoupled position. The dose selector clutch may in this configuration be part of the rotational dose selector.

Alternatively or additionally, the push button, the dose selector and the clutch may be combined in one part.

In one or more examples, the dose delivery device further comprises a spring configured to interact with the clutch. The spring may be tensioned when the clutch is displaced in the axial direction from the coupled position to the decoupled position. Upon releasing the pressure applied in the axial direction for tensioning the spring, the spring may return to its original position and thereby displace the clutch from the decoupled position to the coupled position. When the decoupled position is a dose delivery position, the spring may be tensioned during dose delivery and after end of dose delivery, the spring may return the clutch to the coupled position making the dose delivery device ready for dose setting again.

In one or more examples, the housing comprises an inner housing thread in threaded connection with a dosage drum thread on the dosage drum. By relative rotation between the dosage drum and the housing, the dosage drum may be displace relative to the housing in a dose setting direction. The dosage drum and the housing may form a telescopic configuration.

The dose delivery device may comprise a gearing mechanism. In one or more examples, the gearing mechanism comprises one or more of:
a tower comprising an inner tower track and a tower groove;
a driver comprising a driver thread and a driver key;
a dual threaded nut with a first thread engaged with the driver thread for allowing axial and rotational movement of the driver relative to the housing and a second thread engaged with the tower for allowing rotational movement of the tower relative to the housing; and
a dosing nut comprising a dosing nut thread and a dosing nut key engaged with the tower groove.

The piston rod may be arranged in threaded engagement with the dosage nut allowing rotational and axial movement of the dosage nut relative to the housing.

The driver may be rotationally and axially locked to the thrust bearing.

The dual threaded nut may be connected with the dosing nut in a way that allows relative rotational movement but not relative axial movement between the dual threaded nut and the dosing nut.

The dosing nut may be rotationally locked to the tower.

The tower and the driver may be connected via one or more driver keys and the inner tower track. The connection may allow for axial movement of the driver relative to the tower.

In the gearing mechanism, the rotational movement of the driver may cause rotational movement of the tower and the dosing nut. The rotation of the dosing nut may translate into an axial movement of the dosing nut and the dual threaded nut relative to the piston rod such that the driver is moved axially a longer distance than the dosing nut.

In one or more examples of the gearing mechanism in the dose setting mode, a dose can be set by rotating the dose selector, whereby the push button is elevated from one end of the device a distance in the distal direction proportional to the set dose from a position fixed relative to the housing.

In one or more examples of the gearing mechanism in the dose delivery mode, the set dose can then be injected by pressing the push button back to its non-elevated position, through which motion of the push button, the piston rod will move a shorter distance in the proximal direction due to the gearing mechanism than the distance travelled by the push button.

In one or more examples, the tower track is a helical track. In one or more alternative examples, the tower track is a straight track extending in the axial direction.

In one or more examples, the dose delivery device further comprises a cartridge holder, wherein the piston rod is set free to rotate when the cartridge holder is demounted. This allows for a re-use of the dose delivery device by change of the cartridge.

In one or more examples, a piece of the thread path on at least one of the threads on the housing, the driver or the dual threaded nut is substituted by a combination of a straight path along a longitudinal axis of the device and a rotational path, and wherein the end of the rotational path forms the stop of the dose injection.

In one or more examples, the housing encircles the dosage scale drum at least partly, wherein the dosage scale drum comprises dose scale on the exterior and the housing comprises an aperture for displaying a dose value comprised in the dose scale.

Disclosed herein in a fifth aspect is a rotatable dose selector clutch for a dose delivery device. The dose delivery device is having a dosage scale drum with interior dosage scale coupling teeth and a thrust bearing with exterior thrust bearing coupling teeth, the thrust bearing is configured to be threaded coupled to a piston rod.

The dose selector clutch is configured to be arranged in:
a coupled position, wherein the dose selector clutch couples to the dosage scale drum and the thrust bearing, such that the dosage scale drum and the thrust bearing rotates when the dose selector clutch is rotated, and
a decoupled position, wherein the dose selector clutch decouples from the dosage scale drum and the thrust bearing, such that the dosage scale drum and the thrust bearing are free to rotate independently of the dose selector clutch.

The dose selector clutch comprises inner coupling teeth and outer coupling teeth arranged along an annular circumference, the annular circumference extending in a rotational plane perpendicular to a centre axis extending in an axial direction, wherein the inner and outer coupling teeth protrude from the annular circumference with the inner coupling teeth protruding towards the centre axis and the outer coupling teeth protruding away from the centre axis.

Disclosed herein in a sixth aspect is a rotatable dose selector for a dose delivery device having a dosage scale drum with interior dosage scale coupling teeth and a thrust bearing with exterior thrust bearing coupling teeth, the thrust bearing is configured to be threaded coupled to a piston rod.

The dose selector is configured to be arranged in:
a coupled position, wherein the dose selector couples to the dosage scale drum and the thrust bearing when the dose selector is rotated, such that the dosage scale drum and the thrust bearing rotates when the dose selector is rotated,
a decoupled position, wherein the dose selector decouples from the dosage scale drum and the thrust bearing, such that the dosage scale drum and the thrust bearing are free to rotate independently of the dose selector,

The dose selector comprises a clutch comprising inner coupling teeth and outer coupling teeth, arranged along an annular circumference, the annular circumference extending in a rotational plane perpendicular to a centre axis extending in an axial direction, wherein the inner and outer coupling teeth protrude from the annular circumference with the inner coupling teeth protruding towards the centre axis and the outer coupling teeth protruding away from the centre axis,

In the coupled position, the outer coupling teeth are configured to interact with the dosage dial coupling teeth, and the inner coupling teeth are configured to interact with the thrust bearing coupling teeth, when the dose selector is rotated.

Disclosed herein in a seventh aspect is a push button for a dose delivery device having a dosage scale drum with interior dosage scale coupling teeth and a thrust bearing with exterior thrust bearing coupling teeth, the thrust bearing is configured to be threaded coupled to a piston rod.

The push button is configured to be arranged in a
coupled position, wherein the push button couples to the dosage scale drum and the thrust bearing when the push button is rotated, such that the dosage scale drum and the thrust bearing rotates when the push button rotates,
decoupled position, wherein the push button decouples from the dosage scale drum and the thrust bearing, such that the dosage scale drum and the thrust bearing are free to rotate independently of the push button.

The push button comprises a clutch comprising inner coupling teeth and outer coupling teeth arranged along an annular circumference, the annular circumference extending in a rotational plane perpendicular to a centre axis extending in an axial direction, wherein the inner and outer coupling teeth protrude from the annular circumference with the inner coupling teeth protruding towards the centre axis and the outer coupling teeth protruding away from the centre axis,

In the coupled position, the outer coupling teeth are configured to interact with the dosage scale coupling teeth, and the inner coupling teeth are configured to interact with the thrust bearing coupling teeth, when the push button rotates.

### BRIEF DESCRIPTION OF THE DRAWINGS

Various examples are described hereinafter with reference to the figures. Like reference numerals refer to like elements throughout. Like elements will, thus, not be described in detail with respect to the description of each figure. It should also be noted that the figures are only intended to facilitate the description of the examples. They are not intended as an exhaustive description of the claimed invention or as a limitation on the scope of the claimed invention. In addition, an illustrated example needs not have all the aspects or advantages shown. An aspect or an advantage described in conjunction with a particular example is not necessarily limited to that example and can be practiced in any other examples even if not so illustrated, or if not so explicitly described.

FIG. 1 shows one embodiment of the rotatable dose selector clutch.

FIG. 2 shows three embodiments of the dose selector with the dose selector clutch.

FIG. 3 shows one exemplary embodiment of the dosage scale drum and the thrust bearing.

FIG. 4 illustrates one exemplary embodiment of the dose delivery device and of the housing.

FIG. 5 illustrates one exemplary embodiment of the unassembled dose delivery device.

FIG. 6 illustrates one exemplary embodiment of the driver and the tower.

FIG. 7 illustrates one exemplary embodiment of the dual threaded nut and the dosing nut.

FIG. 8 illustrates one exemplary embodiment of the click bushing, piston rod and foot.

FIGS. 9 and 10 illustrates one exemplary embodiment of the dose delivery device when operated in dose setting mode and in dose delivery mode.

### DESCRIPTION OF EXAMPLES

Exemplary examples will now be described more fully hereinafter with reference to the accompanying drawings. In this regard, the present examples may have different forms and should not be construed as being limited to the descriptions set forth herein. Accordingly, the examples are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

In the drawings, thicknesses of a plurality of layers and areas are illustrated in an enlarged manner for clarity and ease of description thereof. When a layer, area, element, or plate is referred to as being “on” another layer, area, element, or plate, it may be directly on the other layer, area, element, or plate, or intervening layers, areas, elements, or plates may be present there between. Conversely, when a layer, area, element, or plate is referred to as being “directly on” another layer, area, element, or plate, there are no intervening layers, areas, elements, or plates there between. Further when a layer, area, element, or plate is referred to as being “below” another layer, area, element, or plate, it may be directly below the other layer, area, element, or plate, or intervening layers, areas, elements, or plates may be present there between. Conversely, when a layer, area, element, or plate is referred to as being “directly below” another layer, area, element, or plate, there are no intervening layers, areas, elements, or plates there between.

The spatially relative terms “lower” or “bottom” and “upper” or “top”, “below”, “beneath”, “less”, “above”, and the like, may be used herein for ease of description to describe the relationship between one element or component and another element or component as illustrated in the drawings. It will be understood that the spatially relative terms are intended to encompass different orientations of the device in use or operation, in addition to the orientation depicted in the drawings. For example, in the case where a device illustrated in the drawings is turned over, elements described as being on the “lower” side of other elements, or “below” or “beneath” another element would then be oriented on “upper” sides of the other elements, or “above” another element. Accordingly, the illustrative term “below” or “beneath” may include both the “lower” and “upper” orientation positions, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below, and thus the spatially relative terms may be interpreted differently depending on the orientations described.

Throughout the specification, when an element is referred to as being “connected” to another element, the element is “directly connected” to the other element, or “electrically connected” to the other element with one or more intervening elements interposed there between.

The terminology used herein is for the purpose of describing particular examples only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that, although the terms “first,” “second,” “third,” and the like may be used herein to describe various elements, these elements should not be limited by these terms. These terms are only used to distinguish one element from another element. Thus, “a first element” discussed below could be termed “a second element” or “a third element,” and “a second element” and “a third element” may be termed likewise without departing from the teachings herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

Exemplary examples are described herein with reference to cross section illustrations that are schematic illustrations of idealized examples, wherein like reference numerals refer to like elements throughout the specification. As such, variations from the shapes of the illustrations as a result, for example, of manufacturing techniques and/or tolerances, are to be expected. Thus, examples described herein should not be construed as limited to the particular shapes of regions as illustrated herein but are to include deviations in shapes that result, for example, from manufacturing. For example, a region illustrated or described as flat may have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims. Some of the parts, which are not associated with the description may not be provided in order to specifically describe exemplary examples of the present disclosure.

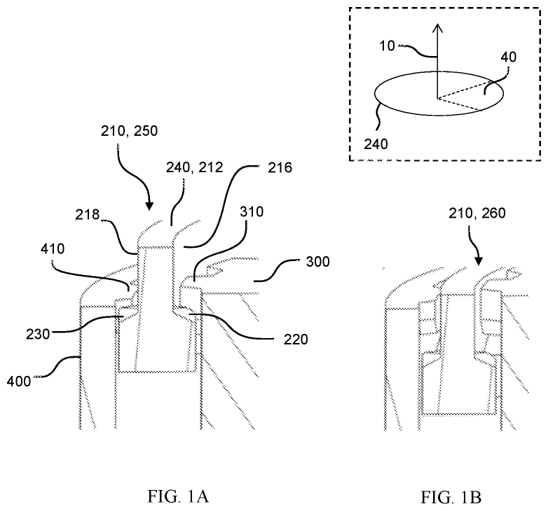

FIG. 1 illustrates one exemplary embodiment of the rotatable dose selector clutch 210. The clutch 210 is configured for interacting with a dosage scale drum 400 and a thrust bearing 300 via coupling means. The clutch 210 is configured to be in a coupled position 250 as illustrated in FIG. 1A or in a decoupled position 260 as illustrated in FIG. 1B.

The rotatable dose selector clutch 210 is arranged between the dosage scale drum 400 and the thrust bearing 300.

The dose selector clutch 210 comprises inner coupling means 220 and outer coupling means 230 arranged along the annular circumference 240. The annular circumference 240 extends in a rotational plane 40 perpendicular to a longitudinal axis 10 extending in an axial direction; this is illustrated in the insert in the upper right-hand part of FIG. 1.

In the illustrated embodiment in FIG. 1, the clutch 210 comprises a tubular part 212 constituting the annular circumference 240 and the inner and outer coupling means 220, 230 in the form of ratchet teeth protruding from the tubular part 212 in opposite directions but in the same rotational plane. Hence, the ratchet teeth 220, 230 extend perpendicular to the annular circumference 240 with the inner coupling means 220 protruding towards the centre axis 10 and the outer coupling means 230 protruding away from the centre axis 10. The inner coupling means 220 protrudes from an inner surface 216 of the tubular part 212 and the outer coupling means 230 protrude from an outer surface 218 of the tubular part 212.

The dose selector clutch 210 is configured to be arranged in:
the coupled position 250, as illustrated in FIG. 1A, wherein the dose selector clutch 210 couples to both the dosage scale drum 400 and the thrust bearing 300, such that the dosage scale drum 400 and the thrust bearing 300 rotates when the dose selector clutch 210 is rotated, and
the decoupled position 260, as illustrated in FIG. 1B, wherein the dose selector clutch 210 decouples from the dosage scale drum 400 and the thrust bearing 300, such that the dosage scale drum 400 and the thrust bearing 300 are free to rotate independently of the dose selector clutch 210.

In the illustrated embodiment in FIG. 1, the dosage scale drum 400 comprises interior dosage scale coupling means 410 in the form of ratchet teeth. The thrust bearing 300 comprises exterior thrust bearing coupling means 310 also in the form of ratchet teeth.

The clutch 210 couples to the dosage scale drum 400 via interaction between the outer coupling means 230 and the interior dosage scale coupling means 410. The clutch 210 couples to the thrust bearing 300 via the inner coupling means 220 and the exterior thrust bearing coupling means 310.

Hence, in the coupled position 250 the outer coupling means 230 and the interior dosage scale coupling means 410 are arranged aligned in one rotational plane. Likewise, the inner coupling means 220 and the exterior thrust bearing coupling means 310 are arranged aligned in one rotational plane. Both set of coupling means, i.e. the first set comprising the outer coupling means 230 and the interior dosage scale coupling means 410, and the second set comprising the inner coupling means 220 and the exterior thrust bearing coupling means 310, can be arranged in the same rotational plane or they can be arrange in two different but parallel rotational planes. The rotational planes may be aligned with the rotational plane 40 or displaced parallel to this plane in the axial direction along the longitudinal axis 10.

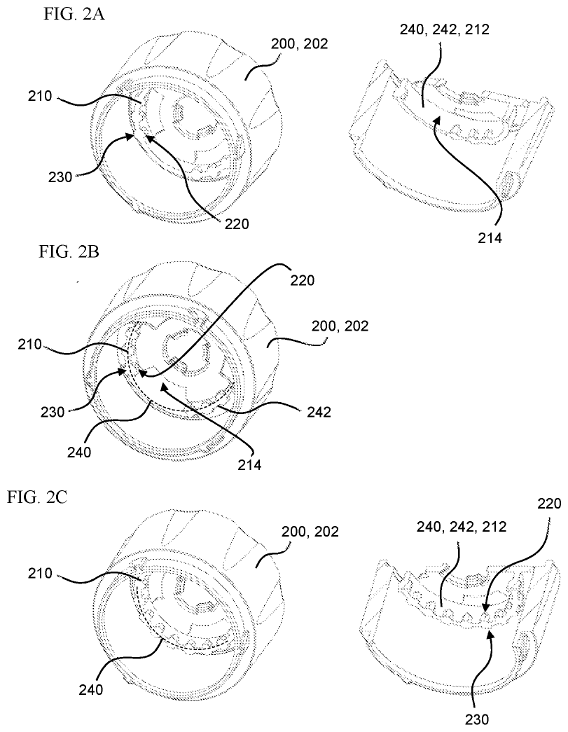

FIG. 2 illustrates three exemplary embodiments of the rotatable dose selector 200 comprising the clutch 210. FIGS. 2A and 2C illustrates the selector 200 in full and as a cut-through part.

FIG. 2A illustrates one embodiment of the dose selector 200 with a clutch 210 comprising inner coupling means 220 and outer coupling means 230 in the form of ratchet teeth. The illustrated clutch furthermore comprises a tubular part 212 arranged along an annular circumference 240. The ratchet teeth are grouped in minor sections along the annular circumference 240 each comprising three teeth protruding in an outward direction and three teeth protruding in an inward direction. The ratchet teeth in the illustrated embodiment protrude from the tubular part 212 perpendicular to the annular circumference 240 with the inner coupling means 220 protruding towards the centre axis 10, and the outer coupling means 230 protruding away from the centre axis 10. The clutch 210 has an annular body 242 extending along the annular circumference 240 with the inner and outer coupling means 220, 230 protruding from the annular body 242. The annular body 242 is formed as a tubular part 212 with an aperture 214 through which at least a part of the thrust bearing 300 can be received. The centre axis 10 is illustrated in FIG. 1.

FIG. 2B illustrates another embodiment of the dose selector 200 with a clutch 210 comprising inner coupling means 220 and outer coupling means 230 in the form of ratchet teeth. The illustrated clutch 210 comprises sectional part arranged along the annular circumference 240 each sectional part comprising three teeth protruding in an outward direction and three teeth protruding in an inward direction. The ratchet teeth in the illustrated embodiment protrude perpendicular to the annular circumference 240 with the inner coupling means 220 protruding towards the centre axis 10, and the outer coupling means 230 protruding away from the centre axis 10. The centre axis 10 is illustrated in FIG. 1.

FIG. 2C illustrates yet another embodiment of the dose selector 200 with a clutch 210 comprising inner coupling means 220 and outer coupling means 230 in the form of ratchet teeth. The embodiment is comparable with that illustrated in FIG. 2A but with inner and outer coupling means arranged continuously spaced along the tubular part 212.

For the illustrated exemplary embodiments of the rotatable dose selector 200, when it comes to the clutch 210, the inner coupling means 220 in the shape of the ratchet teeth may be shifted with respect to the outer coupling means 230 in the shape of the ratchet teeth in the rotational plane 40. Thus, the inner coupling means teeth 220 may protrude inwardly at the same rotational position as the outer coupling means teeth 230 protrude outwardly as shown in FIGS. 2A-C. Alternatively, the ratchet teeth may be protrude inwardly/outwardly in different positions along the rotational plane, such that e.g. one inwardly protruding ratchet tooth is positioned between two outwardly protruding ratchet teeth.

The number of ratchet teeth are illustrated as the same for the inner coupling means 220 and the outer coupling means 230 in FIGS. 2A-C. The number of inner ratchet teeth need, however, not be the same as the number of outer ratchet teeth.

For the illustrated exemplary embodiments of the rotatable dose selector 200, when it comes to the clutch 210, the coupling and de-coupling with the dosage scale drum 400 and the thrust bearing 300, the arrangements and working principle illustrated in FIG. 1 also applies for the three illustrated embodiments in FIG. 2. Likewise, for the illustrated embodiments in FIGS. 2A-2C, when the dose selector 200 is in a coupled position with the dosage scale drum 400 and the thrust bearing 300, both sets of coupling means i.e. the first set comprising the outer coupling means 230 and the interior dosage scale coupling means 410, and the second set comprising the inner coupling means 220 and the exterior thrust bearing coupling means 310 can be arranged in the same rotational plane. Alternatively, they can be arrange in two different but parallel rotational planes. The rotational planes may be aligned with the rotational plane 40 or displaced parallel to this plane in the axial direction along the longitudinal axis.

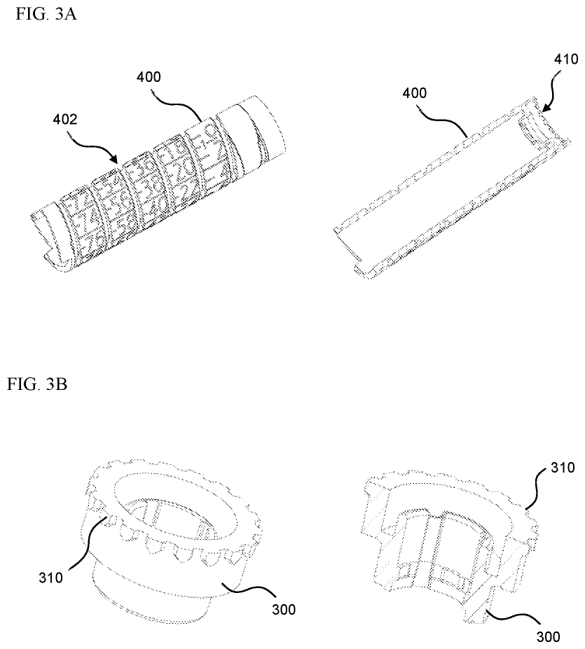

FIG. 3 illustrates one exemplary embodiments of the dosage scale drum 400 in FIG. 3A and the thrust bearing 300 in FIG. 3B. The embodiments are illustrated in full and as cut-through parts.

The cut-through illustration of the dosage scale drum 400 shows the interior dosage scale coupling means 410. In this exemplary embodiment, the coupling means 410 are illustrated as ratchet teeth at the upper-most end of the dosage scale drum 400. The coupling means 410 may be arrange in a lower position, as long as it allows for coupling and decoupling with the outer coupling means 230 of the clutch 210. Likewise, the coupling means 410 may be formed differently than the ratchet teeth as long as they are aimed at the same purpose of coupling and de-coupling to the outer coupling means 230 of the clutch 210.

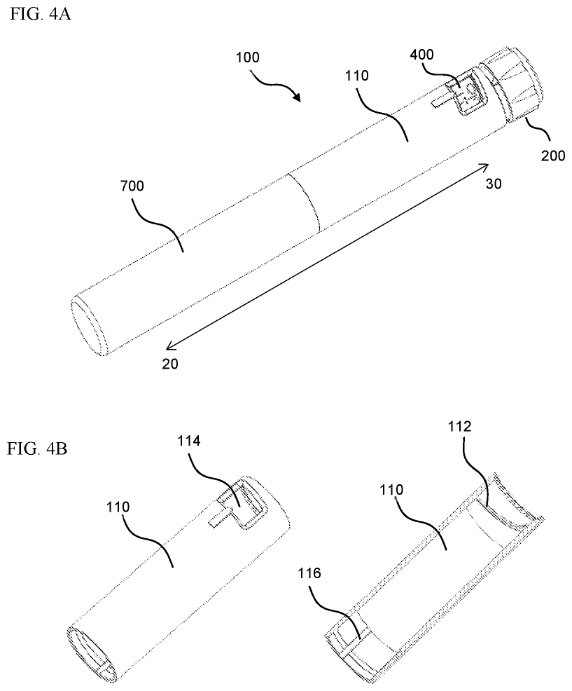

The illustrated dosage drum 400 comprises a dosage drum thread 402 on the exterior side configured to be in threaded communication with a housing 100 via an inner housing thread 112, see FIG. 4.

The illustrated embodiment of the thrust bearing 300 comprises the exterior thrust bearing coupling means 310 at the upper-most end of the thrust bearing 300. The coupling means 310 may be arranged in a lower position, as long as it allows for coupling and decoupling with the inner coupling means 220 of the clutch 210.

The coupling means 310 are illustrated as ratchet teeth, but may be formed differently aimed at the same purpose of coupling and de-coupling to the inner coupling means 220 of the clutch 210.

FIG. 4A illustrates one exemplary embodiment of the dose delivery device 100. The illustrated embodiment shows the dose delivery device 100 as seen from the outside in an assembled state. The illustrated delivery device 100 comprises a cap 700 the housing 110 with an opening 114 through which opening, the scale drum 400 is partly visible, and the dose selector 200 for setting the dose by rotation of the dose selector 200.

The dose selector 200 may also be referred to as a dose knob. The dosage scale drum 400 rotates with the dose selector 200 when setting the dose due to the coupled connection with the clutch 210 (not shown in FIG. 4A). The opening in the housing displays a value of the set dose due to the rotation of the dosage drum 400.

FIG. 4B illustrate one exemplary embodiment of the housing 110 in full and as a cut-through part. The housing 110 comprises an internal thread 112. adapted to be in correspondence with the threading on the exterior of the dosage scale drum 400, see FIG. 3A. The inner housing thread 112 is configured to be in threaded connection with a dosage drum thread 402.

The dosage drum 400 is rotated in the dose setting mode due to the coupling to the rotatable dose selector clutch 210. However, in dose delivery mode, when the clutch 210 is decoupled from the dosage drum 400, the dosage drum 400 may be rotated due to this threaded communication with the housing 110. The rotation of the dosage drum 400 in dose delivery mode may cause the dosage drum 400 to return to it's original position after dose delivery, such that the displayed value in the opening 144 in the house 110 returns to the initial value, e.g. 0, for a non-set dose.

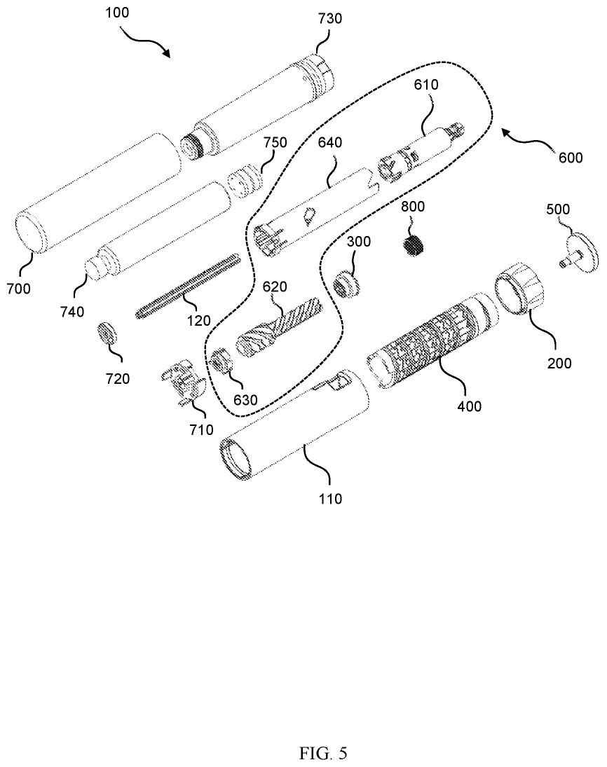

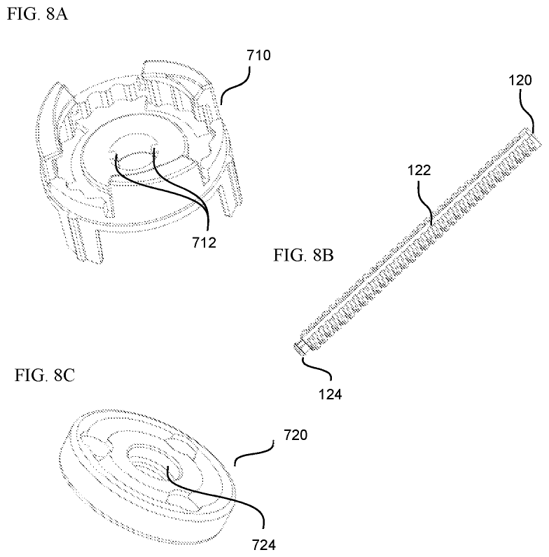

Inside the housing 100 is a housing track 116, which normally rotationally locks a piston click bushing 710 (see FIGS. 5 and 8) to the housing 110.

FIG. 5 illustrates one exemplary embodiment of the dose delivery device 100 according to the invention in an exploded view. The clutch 210 is not visible in the figure.

The parts illustrated in FIG. 5 includes among others the housing 110, the dosage scale drum 400, the thrust bearing 300, a tower 640 and a piston rod 120.

The parts may furthermore include the dosage selector 200, the push button 500, a spring 800, a foot 720, a cartridge holder 730 and a cap 700 for covering the cartridge holder 730. The cartridge holder 730 will normally be adapted for containing a cartridge 740 containing the medicament and a stopper 750. The medicament is pushed out of the cartridge 740 by moving the stopper 750 in a proximal direction.

The parts may also include the piston click bushing 710, which is normally rotationally locked to the housing 110. The piston click bushing 710 is also normally locked to the housing 110 in the longitudinal direction.

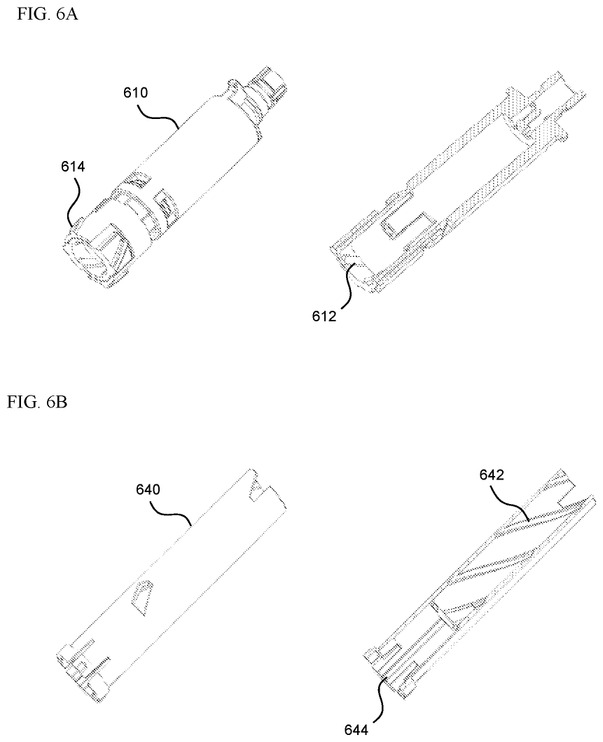

To achieve a gearing mechanism 600, the tower 640 may comprise an inner tower track 642 and a tower groove 644. The inner tower track 642 may be formed as a helical track as illustrated in FIG. 6B or as a straight track extending in the axial direction.

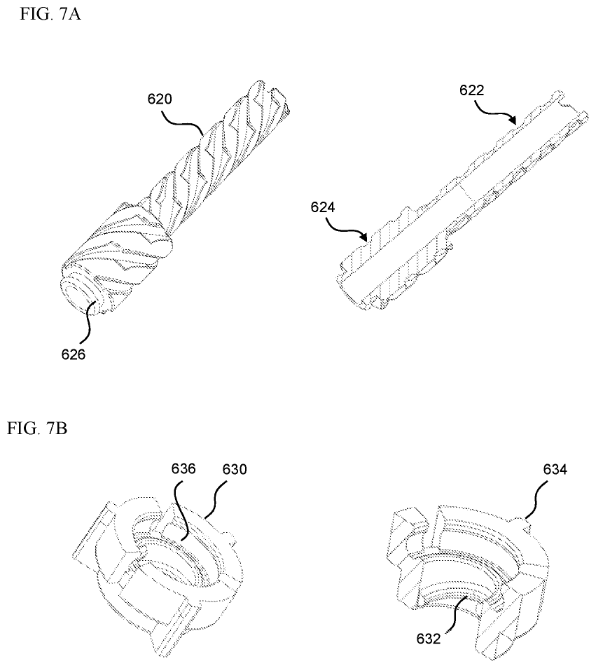

In addition, to achieve the gearing mechanism 600, the dose delivery device may further include the driver 610 comprising a driver thread 612, see FIG. 6A, the dual threaded nut 620 with a first and a second thread 622, 624 (see FIG. 7A) and the dosing nut 630 comprising the dosing nut thread 632 and the dosing nut key 634 (see FIG. 7B).

The working principle of the gearing is that by rotation of the driver 610, the driver 610 is rotationally and axially moved along the first thread 622 of the dual threaded nut 620. In addition, the rotation of the driver 610 also causes the tower 640 to rotate due to the engagement between the tower track 642 and the driver key 614 on the driver 610. This rotation of the tower 640 results in the dosing nut 630 being rotated due to rotational engagement between the tower groove 644 on the tower 640 and the dosing nut key 634 of the dosing nut 630. The rotation of the dosing nut 630 caused by the tower 640 rotation causes an axial movement of the dosing nut 630 along the piston rod 120 (see FIG. 8 for an exemplary embodiment of the piston rod 120), since the piston rod 120 is rotationally fixed to the housing 110 and thereby not rotating. The dosing nut 630 comprises an inner dosing nut recess 636, which houses a dual threaded nut ring 626 locking the dosing nut 630 and the dual threaded nut 620 in the longitudinal direction. The axial movement of the dosing nut 630 translate an axial movement of the dual threaded nut 620, due to the coupling of the dosing not 630 and the dual threaded nut 620 in the longitudinal direction. This again causes the driver 610 to gain additional axial movement, such that the driver 610 is moved axially a longer distance than the dosing nut 630 when setting the dose.

By coupling the dose selector 200 to the driver 610 via the clutch 210 and the thrust bearing 300, a dose can be set in the dose setting mode, by rotating the dose selector 200 whereby the push button 500 is elevated from one end of the device a distance in the distal direction 20 proportional to the set dose from a position fixed relative to the housing 110. In the dose delivery mode 104, the set dose can then be injected by pressing the push button 500 back to its non-elevated position. In the dose delivery mode 104, the dosing nut 630 and the tower 640 are not rotating, which forces the piston rod 120 proximally when the dosing nut 630 is pushed proximally. The push button 500 travels the same distance during dose setting in the dose setting mode 120 and during dose delivery in the dose delivery mode 104 when reaching the non-elevated position. Due to the gearing mechanism 600, the piston rod 120 moves a shorter distance in the proximal direction 30 than the distance travelled by the push button 500.

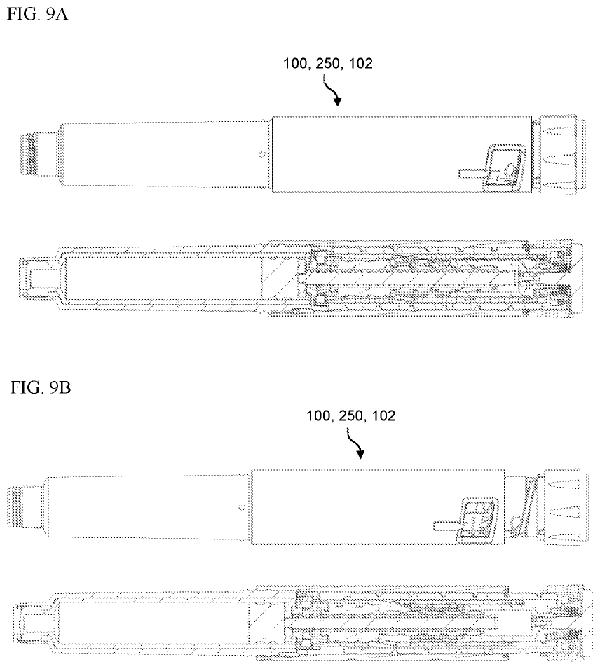

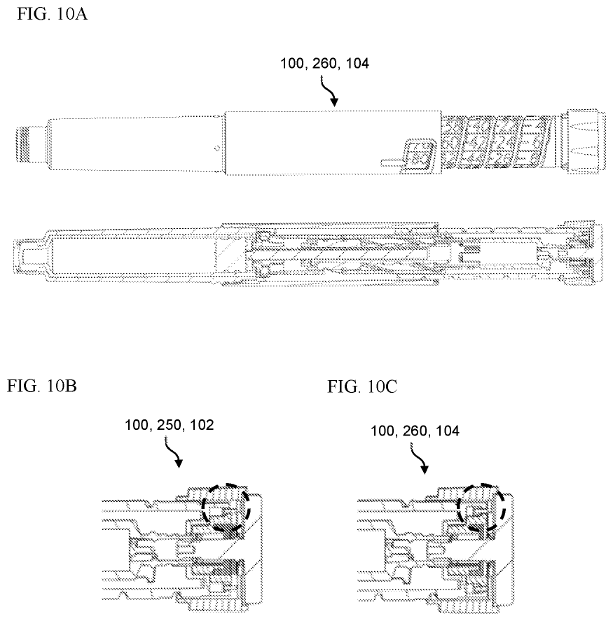

The arrangement of the parts in the assembled dose delivery device and the working principle of the dose delivery device with the gearing mechanism is illustrated in more detail in FIGS. 9 and 10.

FIG. 6 illustrates one exemplary embodiment of the driver 610 and the tower 640.

FIG. 6A illustrate one exemplary embodiment of the driver 610 in full and as a cut-through part. The illustrated driver 610 comprises a driver key 614 adapted to be in communication with the inner tower track 642. The illustrated driver 610 furthermore comprises a driver thread 612 adapted to be in threaded connection with the first thread 622 of the dual threaded nut 620, see FIG. 7A for an exemplary embodiment of the dual threaded nut 620.

FIG. 6B illustrate one exemplary embodiment of the tower 640 in full and as a cut-through part. The illustrated tower 640 comprises an inner tower track 642 and a tower groove 644. In this illustrated embodiment, the tower track is formed as a helical track. In another exemplary embodiment of the tower 640, the tower track may be formed as a straight track, not illustrated.

FIG. 7 illustrates one exemplary embodiment of the dual threaded nut 620 and the dosing nut 630.

FIG. 7A illustrate one exemplary embodiment of the dual threaded nut 620 in full and as a cut-through part. The dual threaded nut 620 comprises a first thread 622 and a second thread 624. The first thread 622 may be configured for engagement with the driver thread 612 allowing for axial and rotational movement of the driver 610 relative to the housing, see FIG. 6A for an exemplary embodiment of the driver. The second thread 624 may be configured for engaged with the tower 640 allowing for rotational movement of the tower relative to the housing, see FIG. 6B for an exemplary embodiment of the tower.

FIG. 7B illustrate one exemplary embodiment of the dosing nut 630 in full and as a cut-through part. The dosing nut 630 comprises a dosing nut thread 632 and a dosing nut key 634. The dosing nut thread 632 is configured for threaded engagement with the piston rod 120, see FIG. 8B for an exemplary embodiment of the piston rod. The dosing nut thread 632 allows for rotational and axial movement of the dosage nut 630 relative to the piston rod 120.

The dosing nut key 634 is configured for rotational engagement with the tower groove 644.

The dosing nut 630 may be axially fixed to the dual threaded nut 620. In the exemplary embodiment in FIG. 7, the dual threaded nut 620 comprises a circular protrusion 626, i.e. a dual threaded nut ring, below the part comprising the second thread 624, and the dosing nut 630 comprises a circular recess 636 above the dosing nut thread 632. The dosing nut 630 and the dual threaded nut 620 may be axially fixed to each other by e.g. clicking the protrusion 626 of the dual threaded nut 620 into the recess 636 of the dosing nut 630.

By only axially fixing the dosing nut to the dual threaded nut 620 the gearing between the driver 610 and the piston rod 120 is achieved.

FIG. 8 illustrates one exemplary embodiment of the piston click bushing 710, the piston rod 120 and the foot 720.

The piston click bushing 710 may provide for multiple functions. One functionality may be to guide the piston rod 120 in a rotationally locked position. This may be achieved by a centre aperture with a shape allowing only axial movement of the piston rod. In the illustrated embodiment in FIG. 8A this is accomplished by two protrusions 712 in the centre hole adapted to accommodate the diagonal cross-cuts 122 of the illustrated piston rod 120 in FIG. 8B.

The foot 720 is adapted to be mounted on the piston rod 120 to interact with a stopper 850 inside a cartridge 810 comprising the drug to be delivered. The foot 720 may be axially fixed to the piston rod 120. In the exemplary embodiment in FIG. 8, the piston rod 120 comprises a circular piston rod protrusion 124 below the threaded part, and the foot 720 comprises a foot recess 724. The piston rod 120 and the foot 720 may be axially fixed to each other by e.g. clicking the protrusion 124 of the piston rod 120 into the recess 724 of the foot 720.

FIGS. 9 and 10 illustrate one exemplary embodiment of the dose delivery device 100 when operated in dose setting mode 102 and in dose delivery mode 104.

In FIG. 9A the dose delivery device 100 is illustrated in dose setting mode 102 with the dose setting clutch in a coupled position 250. The dose has not been set in the illustrated embodiment, which is illustrated by the displayed value of 0 in the aperture or window of the housing.

FIG. 9B illustrates the dose delivery device 100 during dose setting i.e. in dose setting mode 102 with the dose setting clutch in a coupled position 250. The dose is in the process of being set, which is illustrated by the displayed value of 18 in the aperture or window of the housing.

In FIG. 10A the dose delivery device 100 is illustrated in dose delivery mode 104 with the dose setting clutch in a decoupled position 260. The dose to be delivered has been set in the illustrated embodiment, which is illustrated by the displayed value of 80 in the aperture or window of the housing.

FIGS. 10B and 10C illustrate a close-up of the dose the dose selector clutch of the illustrated embodiment of the dose delivery device 100. FIG. 10B illustrates the clutch in the coupled position 250 and FIG. 10 C illustrates the clutch in the decoupled position 260, see the dashed circles for the clutch.

1. Rotatable dose selector clutch for a dose delivery device, the dose delivery device having a dosage scale drum with interior dosage scale coupling means and a thrust bearing with exterior thrust bearing coupling means, the thrust bearing is configured to be threaded coupled to a piston rod,
wherein the dose selector clutch is configured to be arranged in:
a coupled position, wherein the dose selector clutch couples to the dosage scale drum and the thrust bearing, such that the dosage scale drum and the thrust bearing rotates when the dose selector clutch is rotated, and
a decoupled position, wherein the dose selector clutch decouples from the dosage scale drum and the thrust bearing, such that the dosage scale drum and the thrust bearing are free to rotate independently of the dose selector clutch,
wherein the dose selector clutch comprises inner coupling means and outer coupling means arranged along an annular circumference, the annular circumference extending in a rotational plane perpendicular to a centre axis extending in an axial direction, the inner and outer coupling means protrude from the annular circumference with the inner coupling means protruding towards the centre axis and the outer coupling means protruding away from the centre axis.

2. The rotatable dose selector clutch according to claim 1, wherein the coupled position is adapted for dose setting operation of the dose delivery device and the decoupled position is adapted for dose delivery operation of the dose delivery device.

3. The rotatable dose selector clutch according to claim 1, wherein the inner coupling means, the outer coupling means or both comprises spines and/or ratchet teeth.

4. The rotatable dose selector clutch according to claim 1, wherein the inner coupling means and the outer coupling means protrudes in the same plane in opposite directions.

5. The rotatable dose selector clutch according to claim 1, comprising a second set of outer coupling means configured for interacting with a rotational dose selector for setting the dose.

6. The rotatable dose selector clutch according to any-ene-or-more-of claim 1, wherein the dose selector clutch is part of a rotational dose selector for setting the dose.

7. The rotatable dose selector clutch according to claim 1, comprising an annular body extending along the annular circumference with the inner and outer coupling means protruding from the annular body.

8. The rotatable dose selector clutch according to claim 7, wherein the annular body is formed as a tubular part with an aperture, the tubular part is adapted to receive at least a part of the thrust bearing through the aperture in the axial direction, and the inner and outer coupling means are arranged to protrude from the tubular part.

9. The rotatable dose selector clutch according to claim 8, wherein the inner coupling means, the outer coupling means or both are arranged on a first half part of the tubular part being closest to the aperture adapted to receive at least a part of the thrust bearing.

10. The rotatable dose selector clutch according to claim 8, wherein the tubular part comprises an inner surface and an outer surface, wherein the inner coupling means protrude from the inner surface, and the outer coupling means protrude from the outer surface.

11. The rotatable dose selector clutch according to claim 5, wherein the dose selector has an outer surface for being manually rotatable operated for setting a dose of the dose delivery device.

12. Rotatable dose selector clutch for a dose delivery device, the dose delivery device having a dosage scale drum with interior dosage scale coupling teeth and a thrust bearing with exterior thrust bearing coupling teeth, the thrust bearing is configured to be threaded coupled to a piston rod,
wherein the dose selector clutch is configured to be arranged in:
a coupled position, wherein the dose selector clutch couples to the dosage scale drum and the thrust bearing, such that the dosage scale drum and the thrust bearing rotates when the dose selector clutch is rotated, and
a decoupled position, wherein the dose selector clutch decouples from the dosage scale drum and the thrust bearing, such that the dosage scale drum and the thrust bearing are free to rotate independently of the dose selector clutch,
wherein the dose selector clutch comprises inner coupling teeth and outer coupling teeth arranged along an annular circumference, the annular circumference extending in a rotational plane perpendicular to a centre axis extending in an axial direction, wherein the inner and outer coupling teeth protrude from the annular circumference with the inner coupling teeth protruding towards the centre axis and the outer coupling teeth protruding away from the centre axis.

13. Rotatable dose selector for a dose delivery device, the dose delivery device having a dosage scale drum with interior dosage scale coupling means and a thrust bearing with exterior thrust bearing coupling means, the thrust bearing is configured to be threaded coupled to a piston rod,
wherein the dose selector is configured to be arranged in:
a coupled position, wherein the dose selector couples to the dosage scale drum and the thrust bearing when the dose selector is rotated, such that the dosage scale drum and the thrust bearing rotates when the dose selector is rotated,
a decoupled position, wherein the dose selector decouples from the dosage scale drum and the thrust bearing, such that the dosage scale drum and the thrust bearing are free to rotate independently of the dose selector,
wherein the dose selector comprises a clutch comprising inner coupling means and outer coupling means arranged along an annular circumference, the annular circumference extending in a rotational plane perpendicular to a centre axis extending in an axial direction, the inner and outer coupling means protrude from the annular circumference with the inner coupling means protruding towards the centre axis and the outer coupling means protruding away from the centre axis,
wherein in the coupled position, the outer coupling means are configured to interact with the dosage dial coupling means, and the inner coupling means are configured to interact with the thrust bearing coupling means, when the dose selector is rotated.

14. The rotatable dose selector according to claim 13, wherein the coupled position is adapted for dose setting operation of the dose delivery device and the decoupled position is adapted for dose delivery operation of the dose delivery device.

15. The rotatable dose selector according to claim 13, wherein the inner coupling means, the outer coupling means or both comprises splines and/or ratchet teeth.

16. The rotatable dose selector according to claim 13, comprising an annular body extending along the annular circumference with the inner and outer coupling means protruding from the annular body.

17. The rotatable dose selector clutch according to claim 16, wherein the annular body is formed as a tubular part with an aperture, the tubular part is adapted to receive at least a part of the thrust bearing through the aperture in the axial direction, and the inner and outer coupling means are arranged to protrude from the tubular part.

18. The rotatable dose selector according to claim 17, wherein the inner coupling means, the outer coupling means or both are arranged on a first half part of the tubular part being closest to the aperture adapted to receive at least a part of the thrust bearing.

19. The rotatable dose selector clutch according to claim 17, wherein the tubular part comprises an inner surface and an outer surface, wherein the inner coupling means protrude from the inner surface, and the outer coupling means protrude from the outer surface.

20. The rotatable dose selector according to claim 13, having an outer surface for being manually rotatable operated for setting a dose of the dose delivery device.

21. Rotatable dose selector for a dose delivery device, the dose delivery device having a dosage scale drum with interior dosage scale coupling teeth and a thrust bearing with exterior thrust bearing coupling teeth, the thrust bearing is configured to be threaded coupled to a piston rod,
wherein the dose selector is configured to be arranged in:
a coupled position, wherein the dose selector couples to the dosage scale drum and the thrust bearing when the dose selector is rotated, such that the dosage scale drum and the thrust bearing rotates when the dose selector is rotated,
a decoupled position, wherein the dose selector decouples from the dosage scale drum and the thrust bearing, such that the dosage scale drum and the thrust bearing are free to rotate independently of the dose selector,
wherein the dose selector comprises a clutch comprising inner coupling teeth and outer coupling teeth, arranged along an annular circumference, the annular circumference extending in a rotational plane perpendicular to a centre axis extending in an axial direction, wherein the inner and outer coupling teeth protrude from the annular circumference with the inner coupling teeth protruding towards the centre axis and the outer coupling teeth protruding away from the centre axis,
wherein in the coupled position, the outer coupling teeth are configured to interact with the dosage dial coupling teeth, and the inner coupling teeth are configured to interact with the thrust bearing coupling teeth, when the dose selector is rotated.

22. Push button for a dose delivery device, the dose delivery device having a dosage scale drum with interior dosage scale coupling means and a thrust bearing with exterior thrust bearing coupling means, the thrust bearing is configured to be threaded coupled to a piston rod,
wherein the push button is configured to be arranged in:
a coupled position, wherein the push button couples to the dosage scale drum and the thrust bearing when the push button is rotated, such that the dosage scale drum and the thrust bearing rotates when the push button rotates,
a decoupled position, wherein the push button decouples from the dosage scale drum and the thrust bearing, such that the dosage scale drum and the thrust bearing are free to rotate independently of the push button,
wherein the push button comprises a clutch comprising inner coupling means and outer coupling means arranged along an annular circumference, the annular circumference extending in a rotational plane perpendicular to a centre axis extending in an axial direction, the inner and outer coupling means protrude from the annular circumference with the inner coupling means protruding towards the centre axis and the outer coupling means protruding away from the centre axis,
wherein in the coupled position, the outer coupling means are configured to interact with the dosage scale coupling means, and the inner coupling means are configured to interact with the thrust bearing coupling means, when the push button rotates.

23. The push button according to claim 22, wherein the coupled position is adapted for dose setting operation of the dose delivery device and the decoupled position is adapted for dose delivery operation of the dose delivery device.

24. The push button according to claim 22, wherein the inner coupling means, the outer coupling means or both comprises splines and or ratchet teeth.

25. The push button according to claim 22, wherein the clutch comprises an annular body extending along the annular circumference with the inner and outer coupling means protruding from the annular body.

26. The push button according to claim 25, wherein the annular body is formed as a tubular part with an aperture, the tubular part is adapted to receive at least a part of the thrust bearing through the aperture in the axial direction, and the inner and outer coupling means are arranged to protrude from the tubular part.

27. The push button according to claim 26, wherein the inner coupling means, the outer coupling means or both are arranged on a first half part of the tubular part being closest to the aperture adapted to receive at least a part of the thrust bearing.

28. The push button according to claim 26, wherein the tubular part comprises an inner surface and an outer surface, wherein the inner coupling means protrude from the inner surface, and the outer coupling means protrude from the outer surface.

29. The push button according to claim 22, wherein the clutch comprises an outer surface for being manually rotatable operated for setting a dose of the dose delivery device, such that the push button is configured as a dose setting knob.

30. Push button for a dose delivery device, the dose delivery device having a dosage scale drum with interior dosage scale coupling teeth and a thrust bearing with exterior thrust bearing coupling teeth, the thrust bearing is configured to be threaded coupled to a piston rod,
wherein the push button is configured to be arranged in:
a coupled position, wherein the push button couples to the dosage scale drum and the thrust bearing when the push button is rotated, such that the dosage scale drum and the thrust bearing rotates when the push button rotates,
a decoupled position, wherein the push button decouples from the dosage scale drum and the thrust bearing, such that the dosage scale drum and the thrust bearing are free to rotate independently of the push button,
wherein the push button comprises a clutch comprising inner coupling teeth and outer coupling teeth arranged along an annular circumference, the annular circumference extending in a rotational plane perpendicular to a centre axis extending in an axial direction, wherein the inner and outer coupling teeth protrude from the annular circumference with the inner coupling teeth protruding towards the centre axis and the outer coupling teeth protruding away from the centre axis,
wherein in the coupled position, the outer coupling teeth are configured to interact with the dosage scale coupling teeth, and the inner coupling teeth are configured to interact with the thrust bearing coupling teeth, when the push button rotates.

31. A dose delivery device comprising a rotatable dose selector clutch according to a claim 1, wherein the dose delivery device further comprises:
a housing;
a tower;
a dosage scale drum comprising dosage scale coupling means;
a thrust bearing comprising thrust bearing coupling means;
a piston rod arranged in a longitudinal axial direction and rotationally locked to the housing and in threaded engagement with the thrust bearing allowing axial movement of the thrust bearing relative to the housing,
wherein the dose delivery device is configured to be operated in:
a dose setting mode with the rotatable clutch arranged in a coupled position, wherein said outer coupling means are configured to interact with the dosage dial coupling means, and said inner coupling means are configured to interact with the thrust bearing coupling means, when the dose selector clutch is rotated, such that the dosage scale drum, the thrust bearing and the dose selector clutch are rotationally locked to each other and are axially displaced relative to the piston rod in a distal direction, such that a dosage is set by the thrust bearing's axial position on the piston rod, and
a dose delivery mode with the rotatable dose selector clutch arranged in a decoupled position, wherein the dose selector clutch rotationally decouples from the dosage scale drum and the thrust bearing, and wherein the thrust bearing is axially displaceable in a proximal direction, such that the set dose is delivered by axially displacing the piston rod.

32. The dose delivery device according to claim 31 further comprising:
a rotatable dose selector, and
an axially moveable push button in axial connection with the thrust bearing.

33. The dose delivery device according to claim 32, wherein the dose selector is configured to couple to the clutch at least during the dose setting mode and the push button is configured to interact with the clutch during the dose delivery mode to displace the clutch in an axial direction from the coupled position to the decoupled position.

34. The dose delivery device according to claim 32, wherein the dose selector is configured to couple to the clutch at least during the dose setting mode, and wherein the push button is part of the clutch.

35. The dose delivery device according to claim 32, wherein the push button is configured to interact with the clutch during the dose delivery mode to displace the clutch in an axial direction from the coupled position to the decoupled position, and wherein the dose selector clutch is part of the rotational dose selector.

36. The dose delivery device according to claim 32, wherein the push button, the dose selector and the clutch are combined in one part.

37. The dose delivery device according to claim 32, comprising a spring configured to interact with the clutch wherein the spring is tensioned when the clutch is displaced in the axial direction from the coupled position to the decoupled position.

38. The dose delivery device according to claim 32, wherein the housing comprises an inner housing thread in threaded connection with a dosage drum thread on the dosage drum.

39. The dose delivery device according to claim 31 with a gearing mechanism, the gearing mechanism comprising:
a tower comprising an inner tower track and a tower groove;
a driver comprising a driver thread and a driver key;
a dual threaded nut with a first thread engaged with the driver thread for allowing axial and rotational movement of the driver relative to the housing and a second thread engaged with the tower for allowing rotational movement of the tower relative to the housing;
a dosing nut comprising a dosing nut thread and a dosing nut key engaged with the tower groove,
wherein the piston rod is arranged in threaded engagement with the dosage nut allowing rotational and axial movement of the dosage nut relative to the housing;
wherein the driver is rotationally and axially locked to the thrust bearing;
wherein the dual threaded nut is connected with the dosing nut in a way that allows relative rotational movement but not relative axial movement between the dual threaded nut and the dosing nut;
wherein the dosing nut is rotationally locked to the tower;
wherein the tower and the driver are connected via one or more driver keys and the inner tower track, which connection allows for axial movement of the driver relative to the tower; and
wherein rotational movement of the driver causes rotational movement of the tower and the dosing nut, which rotation of the dosing nut translate into an axial movement of the dosing nut and the dual threaded nut relative to the piston rod such that the driver is moved axially a longer distance than the dosing nut;
wherein in the dose setting mode, a dose can be set by rotating the dose selector, whereby the push button is elevated from one end of the device a distance in the distal direction proportional to the set dose from a position fixed relative to the housing, and
wherein in the dose delivery mode the set dose can then be injected by pressing the push button back to its non-elevated position, through which motion of the push button, the piston rod will move a shorter distance in the proximal direction due to the gearing mechanism than the distance travelled by the push button.

40. The dose delivery device according to claim 39, wherein the tower track is a helical track.

41. The dose delivery device according to claim 39, wherein the tower track is a straight track extending in the axial direction.

42. The dose delivery device according to claim 31 further comprising a cartridge holder, wherein the piston rod is set free to rotate when the cartridge holder is demounted.

43. The dose delivery device according to claim 31, wherein a piece of the thread path on at least one of the threads on the housing, the driver or the dual threaded nut is substituted by a combination of a straight path along a longitudinal axis of the device and a rotational path, and wherein the end of the rotational path forms the stop of the dose injection.

44. The dose delivery device according to claim 31, wherein the housing encircles the dosage scale drum at least partly, wherein the dosage scale drum comprises dose scale on the exterior and the housing comprises an aperture for displaying a dose value comprised in the dose scale.
